(19) United States
(12) Patent Application Publication
(10) Pub. No.: US 2006/0039959 A1
(43) Pub. Date: Feb. 23, 2006

(54) Film-Shaped Mucoadhesive Administration Forms For Administering Cannabis Agents

(76) Inventor: Werner Wessling, Rengsdorf (DE)

Correspondence Address: D Peter Hochberg Company, The Baker Building 6th Floor, 1940 East 6th Street, Cleveland, OH, 44114-2294, US

(21) Appl. No.: 10/517,849

(22) PCT Filed: May 8, 2003
(86) PCT No.: PCT/EP03/04807

§ 371 Date: Jul. 22, 2005

(30) Foreign Application Priority Data
Jun. 14, 2002 (DE) 102 26 494.5

(51) Int. Cl. A61K 36/185; A61L 15/16

(52) U.S. Cl. 424/448; 424/774

(57) ABSTRACT

A film-shaped, mucoadhesive administration form having a content of at least one active agent. The active agent is a cannabis agent.

### CROSS REFERENCE TO RELATED APPLICATIONS

This application is a National Stage application of International Application No. PCT/EP03/04807, filed on May 8, 2003, which claims priority of German application number 102 26 494.5, filed on Jun. 14, 2002.

### BACKGROUND OF THE INVENTION

1. Field of the Invention

The present invention relates to film-shaped, mucoadhesive administration forms which have a content of cannabis agents and which are suitable for administration of cannabis agents for therapeutic purposes. The invention further relates to the use of the said administration forms for treating conditions of disease in humans or animals.

2. Description of the Prior Art

The components of the Indian hemp plant (Cannabis sativa L.) have numerous pharmacological effects, of which the psychotropic effect is most widely known. Apart from this, cannabis components also have anti-emetic, anticonvulsive, muscle-relaxing, analgesic, sedative and appetite-increasing effects.

Because of the psychotropic or euphorizing effect and the dependency potential associated therewith, the therapeutic application of cannabis components is subject to severe restrictions.

It has long been known that cannabis components can be used beneficially for treating insomnia, neuralgias and painful rheumatism, as well as gastric and intestinal disorders. A favourable therapeutic effect of cannabis components has furthermore been observed for the following indications:

Conditions of pain in cases of carcinosis and as a result of chemotherapy; conditions of pain and “wasting” syndrome in connection with AIDS; nausea and vomiting as side effects of chemotherapy as well as in connection with AIDS or hepatitis; neuropathic pain; anorexia or cachexia, especially in connection with AIDS or carcinosis in the advanced stages.

A favourable therapeutic effect of cannabis components has also been observed in connection with paralytic symptoms in connection with multiple sclerosis or traumatic transverse lesions; dystonic motor disturbance; bronchial asthma; epileptic attacks or generalized epilepsia; withdrawal symptoms in connection with alcohol dependence, benzodiazepine dependence and opiate dependence; Parkinson's disease; dementia, especially Morbus Alzheimer; nausea; arthritis; glaucoma; migraine; and dysmenorrhoea.

At present, only the synthetically produced cannabis agent R-(6a, 10a)-Δ-9-tetrahydrocannabinol (Dronabinol) is marketable. This isomer of tetrahydrocannabinol (THC) is sold under the product name Marinol. This medicament is administered orally in the form of capsules. Marinol is used for treating severe loss of weight in AIDS patients and cancer patients who, as a result of chemotherapy, suffer from heavy vomiting.

Apart from the aforementioned THC isomer, cannabis extracts and cannabis oils for therapeutic treatment purposes are also suitable. Application is usually effected via the oral route, e.g. in the form of capsules.

Cannabis extracts contain as pharmacologically active ingredients tetrahydrocannabinol (predominantly Δ-9-tetrahydrocannabinol, in small proportion: Δ-8-tetrahydrocannabinol), cannabidiol, cannabinol and cannabichromen. These active agents are also called cannabinoids (see the list “The Merck Index”, 12th ed., 1996, page 285, No. 1794, as well as page 1573, No. 9349).

Oral administration of cannabis agents, especially of R-(6a, 10a)-Δ-9-tetrahydrocannabinol, in the form of capsules, tablets, pills or other solid, oral administration forms, or in the form of orally administered liquid preparations is disadvantageous for a variety of reasons:
Since on use of the aforementioned administration forms, the absorption of the active agent takes place in the gastrointestinal tract, the time of onset of action is delayed. This is disadvantageous especially with respect to the indications mentioned, which generally require a quick onset of action (e.g. pain therapy).
Cannabis agents are at least partially degraded and inactivated during the passage through the stomach and intestines under the influence of acid and enzymes, so that only part of the administered dose is absorbed and is systemically available.
In this connection, unwanted plasma peak values may occur which are frequently the cause of side effects.
In addition, after oral administration a significant portion of the active substance is already metabolised during the first passage through the liver (“first pass effect”).

These disadvantages are particularly important with respect to the acceptance with which these medicaments are met in the above indicated indications. With the mentioned oral administration forms, it is also disadvantageous that patients, in a particular given situation, regard the extended retention, e.g. of a tablet or capsule (filled with an oily solution) in the mouth as particularly unpleasant.

### SUMMARY OF THE INVENTION

It is therefore the object of the present invention to provide an administration form for the administration of cannabis agents which is free from the above-described disadvantages and which stands out in particular for its improved acceptance and compliance, as well as for advantageous pharmacokinetic properties, especially for a rapid onset of action.

This object is achieved by a film-shaped, mucoadhesive administration form having a content of at least one active agent from the group of the cannabis agents, such as a cannabis extract or a cannabis oil.

The object is furthermore achieved by the use of the film-shaped, mucoadhesive administration forms according to the invention in the treatment of diseases and symptoms.

### DETAILED DESCRIPTION OF THE INVENTION

The administration forms according to the invention are applied, in the form of thin, small flat pieces or wafer-shaped objects (“wafers”), to the oral mucosa where they adhere because of their mucoadhesive properties. Application to the oral mucosa is sublingual or buccal. Furthermore, other mucosal surfaces may also be taken into consideration as an application site, e.g. the nasal mucosa.

During the period of application, the cannabis agent(s) contained in the administration form are released into the surrounding saliva and are subsequently absorbed by the oral mucosa (i.e. transmucosally). In the contact area of the application surface, the active agent may also be released directly from the administration form to the oral mucosa. During application, the administration form absorbs saliva and the active substance contained therein gets to the outside by diffusion.

It is advantageous in this connection that the active agent is released into the saliva after only a short time lag, so that the saliva-active agent mixture immediately reaches all areas of the oral mucosa, where it can be absorbed. The amount of saliva in which the released active agent is dissolved or dispersed per unit of time is relatively small and there occurs no hypersalivation so that swallowing of the active agent (involving the mentioned disadvantages of gastrointestinal absorption) is largely excluded.

Since active agent absorption takes place by circumventing the gastrointestinal route, the above-described disadvantages (delayed onset of action, “first pass effect”) of other oral administration forms (e.g. tablets) are avoided.

With the administration forms of the invention, compliance is increased as well, since application requires no special discipline. Due to their small layer thickness the application of the film-shaped administration forms of the present invention is generally not unpleasant by the treated persons.

According to one embodiment, the administration forms of the invention comprise a polymer matrix which serves as an active agent reservoir and has mucoadhesive properties. At least one layer or at least one surface of the administration form possesses mucoadhesive properties. The administration form may consist of one single layer or comprise a plurality of layers. In the case of a multilayer structure, at least one of the layers contains active agent(s).

In the simplest case, an administration form is made up of a mucoadhesive, preferably monolayer polymer matrix containing one or more cannabis agents. The active agent(s) may be present in the administration form in dissolved, dispersed or emulsified form.

The polymer matrix contains one or more polymers which are water-soluble and/or swellable in an aqueous media. By selecting such polymers, it is possible to influence the mucoadhesive properties and the release behaviour.

Polymers of the following group are particularly suitable as water-soluble or swellable polymers: starch and starch derivatives, dextran; cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl cellulose or propyl cellulose; polyacrylic acid, polyacrylates, polyvinyl pyrrolidones, polyethylene oxide polymers, polyacrylamides, polyethylene glycol, gelatine, collagen, alginates, pectins, pullulan, tragacanth, chitosan, alginic acid, arabinogalactan, galactomannan, agar-agar, agarose, carrageenan, and natural gums.

The polymer portion is 5 to 95%-wt, especially 15 to 75%-wt, relative to the dry matter of the administration form.

According to one embodiment, the administration forms according to the invention contain a cannabis extract or a cannabis oil, in an amount of 0.5 to 50%-wt, especially in an amount of 1 to 30%-wt. Processes for the manufacture of pharmaceutically acceptable cannabis extracts or cannabis oils are known to those skilled in the art.

The invention furthermore comprises administration forms of the mentioned type containing at least one cannabinoid active agent from the group consisting of tetrahydrocannabinol, cannabinol, cannabidiol, and cannabichromen. Tetrahydrocannabinol, especially R-(6a,10a)-Δ-9-tetrahydrocannabinol, is also a suitable active agent. The cannabinoid active agents may be of natural, partially synthetic or synthetic origin.

The active substance content amounts to 0.1 to 20%-wt, especially preferably 0.5 to 10%-wt, relative to the dry matter of an administration form.

An individual administration form contains 0.5 to 20 mg, especially preferably 1 to 10 mg of active agent, e.g. tetrahydrocannabinol.

Optionally, the administration forms according to the invention may contain one or more additives from the following groups: fillers, colourants, flavourings, aromatics, odorous substances, emulsifiers, plasticizers, sweeteners, preservatives, permeation-enhancing substances, pH regulators and antioxidants. Substances suitable for this purpose are in principle known to the skilled artisan.

The administration form according to the present invention can also include flavourings, odorous substances and aromatics, either alone or in combination. It is, for example, possible to improve the impression of the taste by adding a refreshing flavouring (e.g. menthol, eucalyptol). This simultaneously enables inconspicuous intake of the medicament as it smells like a usual refreshment sweet. It additionally contributes to improving compliance.

Especially suitable are, for example, flavourings and aromatics from the group comprising menthol, eucalyptol, limonene, phenyl ethanol, camphene, pinene, seasoning aromatics such as n-butyl phthalide or cineol, as well as eucalyptus oil and thyme oil, methyl salicylate, turpentine oil, camomile oil, ethyl vanillin, 6-methyl coumarin, citronellol, and acetic acid n-butyl ester.

The inventive administration forms containing cannabis agents are film-shaped, i.e. of a thin and flat shape, for example in the form of thin, small flat pieces or small wafers. These film-shaped plates may be of various geometric shapes, e.g. circular, ellipsoid or elongated.

The thickness of the administration form amounts to 0.01 to 2 mm; or in the range of 0.05 to 0.5 mm. To avoid a foreign body sensation, the layer thickness should be as small as possible (such as smaller than 0.2 mm).

To achieve special effects, the administration forms according to the invention may have a bilayer or monolayer structure. The individual layers may differ in terms of one or more of the following parameters: polymer composition, active substance content, active substance concentration, content of additives.

Due to the already mentioned properties, the cannabis agents-containing administration forms according to the invention can be advantageously employed in the treatment of diseases or symptoms, especially in cases of conditions of pain in cases of carcinosis and as a result of chemotherapy; conditions of pain and “wasting” syndrome in connection with AIDS; nausea and vomiting, especially nausea and vomiting as side effects of a chemotherapy as well as in connection with AIDS or hepatitis; neuropathic pain; anorexia or cachexia, especially in connection with AIDS or carcinosis in the advanced stages; paralytic symptoms in connection with multiple sclerosis or traumatic transverse lesions; dystonic motor disturbance; bronchial asthma; epileptic attacks or generalized epilepsia; withdrawal symptoms in connection with alcohol dependence, benzodiazepine dependence and opiate dependence; Parkinson's disease; dementia, especially Alzheimer's disease; nausea; arthritis; glaucoma; migraine; dysmenorrhoea.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A film-shaped, mucoadhesive administration form containing a cannabis agent selected from the group consisting of cannabis extract and cannabis oil.

2. The administration form according to claim 1, wherein said administration form comprises a polymer matrix, said polymer matrix being an active substance reservoir and having mucoadhesive properties.

3. The administration form according to claim 2, wherein said polymer matrix contains at least one polymer being water-soluble and/or swellable in an aqueous media, said at least one polymer is selected from the group consisting of starch and starch derivatives, dextran, carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl cellulose or propyl cellulose, polyacrylic acid, polyacrylates, polyvinyl pyrrolidones, polyethylene oxide polymers, polyacrylamides, polyethylene glycol, gelatine, collagen, alginates, pectins, pullulan, tragacanth, chitosan, alginic acid, arabinogalactan, galactomannan, agar-agar, agarose, carrageenan and natural gums, and wherein said administration form comprises said at least one polymer at a portion of 5 to 95%-wt.

4. The administration form according to claim 1, wherein said administration form contains said cannabis agent selected from the group consisting of cannabis extract and cannabis oil in an amount of 0.5 to 50%-wt.

5. The administration form according to claim 1, wherein said administration form further contains at least one substance selected from the group consisting of flavourings, odorous substances and aromatics.

6. The administration form according to claim 1, wherein the layer thickness is 0.01 to 2 mm.

7. The administration form according to claim 1, wherein said administration form further contains at least one inactive ingredient selected from the group consisting of fillers, colourants, emulsifiers, plasticizers, sweeteners, preservatives, pH regulators, permeation-enhancing substances, and antioxidants.

8. The administration form according to claim 1, wherein said administration form has a multilayer structure with at least one layer having an active agent content.

9. A method of treating conditions of pain in cases of carcinosis and as a result of chemotherapy; conditions of pain and “wasting” syndrome in connection with AIDS; nausea and vomiting, especially nausea and vomiting as side effects of a chemotherapy as well as in connection with AIDS or hepatitis; neuropathic pain; anorexia or cachexia, especially in connection with AIDS or carcinosis in the advanced stages; paralytic symptoms in connection with multiple sclerosis or traumatic transverse lesions; dystonic motor disturbance; bronchial asthma; epileptic attacks or generalized epilepsia; withdrawal symptoms in connection with alcohol dependence, benzodiazepine dependence and opiate dependence; Parkinson's disease; dementia, especially Alzheimer's disease; arthritis; glaucoma; migraine; dysmenorrhoea, said method comprising the step of:
administering a film-shaped, mucoadhesive administration form containing a cannabis agent selected from the group consisting of cannabis extract and cannabis oil to an inflicted person.

10. A method of treating conditions of pain in cases of carcinosis and as a result of chemotherapy; conditions of pain and “wasting” syndrome in connection with AIDS; nausea and vomiting, especially nausea and vomiting as side effects of a chemotherapy as well as in connection with AIDS or hepatitis; neuropathic pain; anorexia or cachexia, especially in connection with AIDS or carcinosis in the advanced stages; paralytic symptoms in connection with multiple sclerosis or traumatic transverse lesions; dystonic motor disturbance; bronchial asthma; epileptic attacks or generalized epilepsia; withdrawal symptoms in connection with alcohol dependence, benzodiazepine dependence and opiate dependence; Parkinson's disease; dementia, especially Alzheimer's disease; arthritis; glaucoma; migraine; dysmenorrhoea, said method comprising the step of:
administering a film-shaped, mucoadhesive administration form to an afflicted person, said administration form containing a cannabinoid active agent selected from the group of active agents consisting of tetrahydrocannabinol, cannabinol, cannabidiol and cannabichromen.

11. The method according to claim 9, wherein the administration form is a film-shaped, mucoadhesive administration form containing a cannabis agent selected from the group consisting of cannabis extract and cannabis oil, and wherein said administration form comprises a polymer matrix, said polymer matrix being an active substance reservoir and having mucoadhesive properties.

12. The method according to claim 9, wherein the treatment is effected by application of the administration form to the oral mucosa.

13. A medicinal product for treating conditions of pain in cases of carcinosis and as a result of chemotherapy; conditions of pain and “wasting” syndrome in connection with AIDS; nausea and vomiting, especially nausea and vomiting as side effects of a chemotherapy as well as in connection with AIDS or hepatitis; neuropathic pain; anorexia or cachexia, especially in connection with AIDS or carcinosis in the advanced stages; paralytic symptoms in connection with multiple sclerosis or traumatic transverse lesions; dystonic motor disturbance; bronchial asthma; epileptic attacks or generalized epilepsia; withdrawal symptoms in connection with alcohol dependence, benzodiazepine dependence and opiate dependence; Parkinson's disease; dementia, especially Alzheimer's disease; arthritis; glaucoma; migraine; dysmenorrhoea, said medicinal product comprising a film-shaped, muco-adhesive administration form containing a cannabis agent selected from the group consisting of cannabis extract and cannabis oil.

14. A medicinal product for treating conditions of pain in cases of carcinosis and as a result of chemotherapy; conditions of pain and “wasting” syndrome in connection with AIDS; nausea and vomiting, especially nausea and vomiting as side effects of a chemotherapy as well as in connection with AIDS or hepatitis; neuropathic pain; anorexia or cachexia, especially in connection with AIDS or carcinosis in the advanced stages; paralytic symptoms in connection with multiple sclerosis or traumatic transverse lesions; dystonic motor disturbance; bronchial asthma; epileptic attacks or generalized epilepsia; withdrawal symptoms in connection with alcohol dependence, benzodiazepine dependence and opiate dependence; Parkinson's disease; dementia, especially Alzheimer's disease; arthritis; glaucoma; migraine; dysmenorrhoea, said medicinal product comprising a film-shaped, mucoadhesive administration form containing a cannabinoid active agent selected from the group of active agents consisting of tetrahydrocannabinol, cannabinol, cannabidiol and cannabichromen.

15. The medicinal product according to claim 14, wherein the administration form is a film-shaped, mucoadhesive administration form containing a cannabis agent selected from the group consisting of cannabis extract and cannabis oil, and wherein said administration form comprises a polymer matrix, said polymer matrix being an active substance reservoir and having mucoadhesive properties, said reservoir containing said cannabis extract or cannabis oil.

16. The medicinal product according to claim 13, wherein the administration form is an administration form for application on the oral mucosa.

17. The administration form according to claim 3, wherein said administration form comprises said polymer at a portion of 15 to 75%-wt.

18. The administration form according to claim 4, wherein said administration form contains said cannabis agent selected from the group consisting of cannabis extract and cannabis oil in an amount of 1 to 30%-wt.

19. The administration form according to claim 5, wherein said flavourings, odorous substances and aromatics are selected from the group consisting of menthol, eucalyptol, limonene, phenyl ethanol, camphene, pinene, n-butyl phthalide, cineol, eucalyptus oil, thyme oil, methyl salicylate, turpentine oil, camomile oil, ethyl vanillin, 6-methyl coumarin, citronellol, and acetic acid n-butyl ester.

20. (canceled)

21. The administration form according to claim 6, wherein the layer thickness is 0.05 to 0.5 mm.

22. The method according claim 12, wherein the application of the administration form to the oral mucosa is selected from the group consisting of sublingual application and buccal application.

23. The method according to claim 10, wherein the administration form is a film-shaped, mucoadhesive administration form containing a cannabinoid agent selected from the group consisting of tetrahydrocannabinol, cannabinol, cannabidiol and cannabichromen and wherein said administration form comprises a polymer matrix, said polymer matrix being an active substance reservoir and having mucoadhesive properties.

24. The method according to claim 10, wherein the treatment is effected by application of the administration form to the oral mucosa.

25. The method according claim 24, wherein the application of the administration form to the oral mucosa is selected from the group consisting of sublingual application and buccal application.

26. The medicinal product according claim 16, wherein the application of the administration form to the oral mucosa is selected from the group consisting of sublingual application and buccal application.

27. The medicinal product according to claim 14, wherein the administration form is an administration form for application on the oral mucosa.

28. The medicinal product according claim 27, wherein the application of the administration form to the oral mucosa is selected from the group consisting of sublingual application and buccal application.

29. A film-shaped, mucoadhesive administration form containing a cannabis agent selected from the group consisting of cannabis extract and cannabis oil, wherein said administration form comprises a polymer matrix, said polymer matrix being an active substance reservoir and having mucoadhesive properties, wherein said administration form contains said cannabis agent selected from the group consisting of cannabis extract and cannabis oil in an amount of 0.5 to 50%-wt, and wherein the layer thickness is 0.01 to 2 mm.

30. The administration form according to claim 29, wherein said polymer matrix contains at least one polymer being water-soluble and/or swellable in an aqueous media, said at least one polymer is selected from the group consisting of starch and starch derivatives, dextran, carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl cellulose or propyl cellulose, polyacrylic acid, polyacrylates, polyvinyl pyrrolidones, polyethylene oxide polymers, polyacrylamides, polyethylene glycol, gelatine, collagen, alginates, pectins, pullulan, tragacanth, chitosan, alginic acid, arabinogalactan, galactomannan, agar-agar, agarose, carrageenan and natural gums, and wherein said administration form comprises said at least one polymer at a portion of 5 to 95%-wt.

31. The administration form according to claim 30, wherein said administration form has a multilayer structure with at least one layer having an active agent content.

32. The administration form according to claim 29, wherein the administration form is a film-shaped, mucoadhesive administration form containing a cannabinoid agent selected from the group consisting of tetrahydrocannabinol, cannabinol, cannabidiol and cannabichromen and wherein said administration form comprises a polymer matrix, said polymer matrix being an active substance reservoir and having mucoadhesive properties.

33. The medicinal product according to claim 13, wherein said administration form comprises a polymer matrix, said polymer matrix being an active substance reservoir and having mucoadhesive properties, said reservoir containing said active agent selected from the group consisting of tetrahydrocannabinol, cannabinol, cannabidiol and cannabichromen.
